(19) United States
(12) Patent Application Publication
(10) Pub. No.: US 2020/0038246 A1
(43) Pub. Date: Feb. 6, 2020

(54) EYE PROTECTION DEVICE

(71) Applicant: Orange Bright Optics Inc., New Taipei City (TW)

(72) Inventors: Ke-Shu Chin, New Taipei City (TW); Chi-Hua Lee, New Taipei City (TW); Tsung-Wei Chan, New Taipei City (TW); Kuang-Hua Chang, New Taipei City (TW); Chih-Cheng Yang, Taipei City (TW)

(73) Assignee: Orange Bright Optics Inc., New Taipei City (TW)

(21) Appl. No.: 16/051,491

(22) Filed: Aug. 1, 2018

(51) Int. Cl. A61F 9/02; G02B 5/26; G02B 5/28; G02C 7/10

(52) U.S. Cl. CPC A61F 9/022; G02B 5/26; F21V 7/0066; G02C 7/104; G02B 5/285

(57) ABSTRACT

An eye protection device including a first transparent substrate, a second transparent substrate, and a high reflection interference film is provided. The second transparent substrate is opposite to the first transparent substrate. The high reflection interference film is disposed on the first transparent substrate or the second transparent substrate.

### BACKGROUND

### Technical Field

The disclosure relates to a protection device, and more particularly to an eye protection device.

### Description of Related Art

The laser light source used in laser processing has energy concentration property. During laser processing, harmful wavelength beams apart from the laser wavelength are produced due to heat produced by laser processing. When the harmful wavelength beams radiate from a laser processing machine, the beams are likely to enter the human's eyes, causing damage to the eyes. Thus, the operation view windows of traditional laser processing machines or protective goggles worn by operators normally have a medium capable of obstructing beams of specific wavelengths to filter the harmful wavelength beams, so as to prevent direct or indirect harmful wavelength beams from damaging the eyes of the operators in laser processing. However, the medium on the operation view windows or the protective goggles and surfaces of device windows are easily damaged (e.g. being scratched) during cleaning, which affects observation of workpiece by the operators during laser processing. Thus, operation view windows and protective goggles have to be replaced regularly, which causes increase in causing the manufacturing cost.

### SUMMARY

The disclosure provides an eye protection device, which helps to lower the cost of element and device replaced, improves the efficiency in workpiece prepared before processing applied, and effectively obstructs the unnecessary beams to protect the safety of human's eyes.

The eye protection device of the disclosure includes a first transparent substrate, a second transparent substrate, and a high reflection interference film. The second transparent substrate is opposite to the first transparent substrate. The high reflection interference film is disposed on the first transparent substrate or the second transparent substrate.

According to an embodiment of the disclosure, the first transparent substrate and the second transparent substrate are respectively glass.

According to an embodiment of the disclosure, the high reflection interference film is positioned between the first transparent substrate and the second transparent substrate.

According to an embodiment of the disclosure, the high reflection interference film is a multilayer thin film which allows visible light to penetrate through, and the multilayer thin film reflects invisible light and light of laser processing wavelength.

According to an embodiment of the disclosure, there is an air gap between the first transparent substrate and the second transparent substrate.

According to an embodiment of the disclosure, the eye protection device further includes an adhesive layer. The adhesive layer is positioned between the first transparent substrate and the second transparent substrate.

According to an embodiment of the disclosure, the eye protection device further includes a high transmittance anti-reflection film. The high transmittance anti-reflection film is disposed on at least one of the first transparent substrate and the second transparent substrate.

According to an embodiment of the disclosure, the high transmittance anti-reflection film is a filter for filtering invisible light and light of laser processing wavelength.

According to an embodiment of the disclosure, the first transparent substrate has a first interior surface facing the second transparent substrate and a first exterior surface opposite to the first interior surface. The second transparent substrate has a second interior surface facing the first transparent substrate and a second exterior surface opposite to the second interior surface. The eye protection device further includes protection layers. The protection layers are disposed on the first exterior surface and the second exterior surface.

According to an embodiment of the disclosure, the eye protection device is protective goggles or an operation window of a laser processing machine.

According to an embodiment of the disclosure, the eye protection device further includes two pieces of polarizers. The two pieces of polarizers overlap the first transparent substrate and the second transparent substrate, and an angle is formed between absorption axes of the two pieces of polarizers.

To make the aforementioned and other features of the disclosure more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 to FIG. 5 are respectively schematic cross-sectional diagrams of eye protection devices according to a first embodiment to a fifth embodiment of the disclosure.

### DETAILED DESCRIPTION OF DISCLOSED EMBODIMENTS

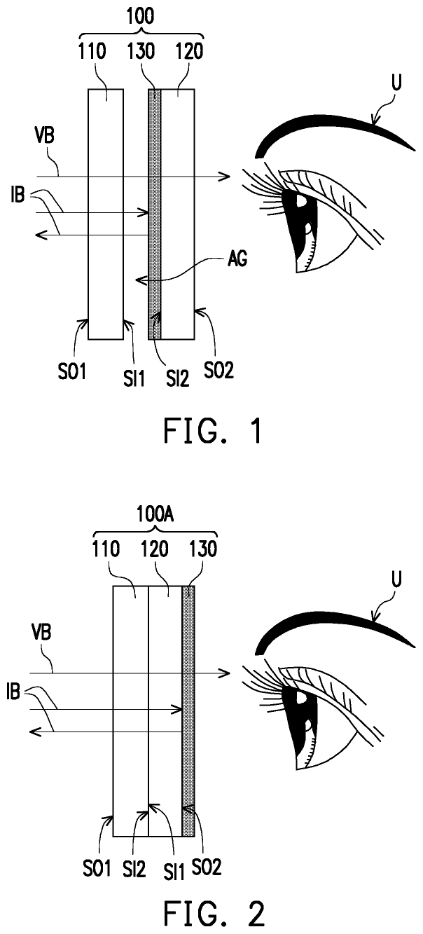

FIG. 1 to FIG. 5 are respectively schematic cross-sectional diagrams of eye protection devices according to a first embodiment to a fifth embodiment of the disclosure, which respectively illustrate five types of eye protection devices of the disclosure. However, persons skilled in the art can make modifications to the embodiments according to needs without departing from the spirit of the disclosure, such as by increasing or decreasing one or more element(s)/film layer(s), and such modifications still fall within the scope to be protected by the disclosure.

In embodiments of the disclosure, the eye protection device can be protective goggles, an operation window of a laser processing machine, or an operation window of other types of machines. For the protective goggles, the eye protection device is worn on the head of the user to protect the eyes of the operator under the environment where harmful wavelength beams exist (e.g., environments of laser processing or other applications of light sources with high intensity and energy concentration). For the operation window of the laser processing machine, the eye protection device is disposed between the operator and the processing machine, and the eye protection device can be viewed by multiple operators at the same time.

Referring to FIG. 1, an eye protection device 100 includes a first transparent substrate 110, a second transparent substrate 120, and a high reflection interference film 130. The second transparent substrate 120 is opposite to the first transparent substrate 110. The high reflection interference film 130 is disposed on the first transparent substrate 110 or the second transparent substrate 120.

Specifically, the first transparent substrate 110 is, for example, a substrate closer to a processing machine or a harmful light source (not shown), while the second transparent substrate 120 is, for example, a substrate closer to an operator U. The first transparent substrate 110 and the second transparent substrate 120 may respectively be glass. At least one of the first transparent substrate 110 and the second transparent substrate 120 may be tempered glass with high mechanical strength, but the disclosure is not limited thereto. In another embodiment, the first transparent substrate 110 and the second transparent substrate 120 may be other suitable substrates, respectively.

The first transparent substrate 110 has a first interior surface SI1 facing the second transparent substrate 120 and a first exterior surface SO1 opposite to the first interior surface SI1. The second transparent substrate 120 has a second interior surface SI2 facing the first transparent substrate 110 and a second exterior surface SO2 opposite to the second interior surface SI2.

The high reflection interference film 130 may be disposed on the first interior surface SI1, the first exterior surface SO1, the second interior surface SI2, or the second exterior surface SO2. Besides, the high reflection interference film 130 is suitable for reflecting beams of specific harmful wavelengths and beams of laser processing wavelengths while allowing beams of other wavelengths to penetrate through. In the embodiment, the high reflection interference film 130 is, for example, a multilayer thin film allowing visible light VB to penetrate through while reflecting ultraviolet light IB (e.g. beams with wavelengths below 400 nm), so that the operator U or other viewers are able to see the workpiece in the laser processing machine and the processing condition thereof. However, the design of the transmission spectrum and the reflection spectrum of the high reflection interference film 130 may be adjusted according to different needs (e.g., wavebands of harmful light sources). For example, the high reflection interference film 130 may be a multilayer interference thin film in which a plurality of high refractive index layers and a plurality of low refractive index layers are alternately arranged. By controlling the refractive index and the thickness of the high refractive index layers and the low refractive index layers, the transmission spectrum and the reflection spectrum of the high reflection interference film 130 may be adjusted.

By positioning the high reflection interference film 130 between the first transparent substrate 110 and the second transparent substrate 120, damages (e.g., scratches) caused to the high reflection interference film 130 during cleaning of the eye protection device 100 (e.g., wiping the first exterior surface SO1 of the first transparent substrate 110 or the second exterior surface SO2 of the second transparent substrate 120) can be prevented, so as to maintain integrity of the high reflection interference film 130. In addition, by the arrangement of two transparent substrates, a damaged transparent substrate (e.g., an operation window of a laser processing machine) can be replaced independently.

In another embodiment, the eye protection device 100 may further include two pieces of polarizers (e.g., high-intensity light adjustable polarizing polarizers, but are not limited thereto) to filter beams of a specific polarization state and high-intensity beams. The two pieces of polarizers overlap the first transparent substrate 110 and the second transparent substrate 120. For example, the two pieces of polarizers may be attached onto at least one of the first interior surface SI1, the first exterior surface SO1, the second interior surface SI2, and the second exterior surface SO2 using an adhesive layer. When the polarizer and the high reflection interference film 130 are formed on the same surface of the transparent substrate, the high reflection interference film 130 may be formed on the surface first before attaching the polarizer, or attaching the polarizer first before forming the high reflection interference film 130. By rotating at least one of the two pieces of polarizers, an angle is formed between the absorption axes of the two pieces of polarizers to adjust (e.g., to lower) light intensity and glare. In an embodiment, at least one of the two pieces of polarizers may be fixed between the operator U and the laser processing machine using a clamping mechanism, so that the at least one of the two pieces of polarizers does not have to be attached onto the first transparent substrate 110 or the second transparent substrate 120.

Since the first transparent substrate 110 is closer to the processing machine, residue caused by laser processing is more likely splashed onto the first exterior surface SO1 of the first transparent substrate 110. In other words, as compared to the second transparent substrate 120, the first transparent substrate 110 is more likely to be damaged (e.g. scratched by residue or scratched due to cleaning). Meaning, the replacement rate of the first transparent substrate 110 is higher. Since the first transparent substrate 110 needs to be replaced more regularly, and the cost of the high reflection interference film 130 is higher than a transparent substrate, disposing the high reflection interference film 130 on the second transparent substrate 120 (e.g., the second interior surface SI2 of the second transparent substrate 120) instead of disposing the high reflection interference film 130 on the first transparent substrate 110 can reduce the number of replacements of the high reflection interference film 130, so as to lower the cost. However, the disclosure is not limited to the above. In another embodiment, the high reflection interference film 130 may also be disposed on the second exterior surface SO2 of the second transparent substrate 120. Alternatively, the high reflection interference film 130 may also be disposed on the first interior surface SI1 (e.g., protective goggles worn by an operator) of the first transparent substrate 110.

In the embodiment, there is an air gap AG between the first transparent substrate 110 and the second transparent substrate 120. Specifically, the first transparent substrate 110 and the second transparent substrate 120 are separated from each other, and the light transmission medium between the first transparent substrate 110 and the high reflection interference film 130 is air. Since the refractive index of the first transparent substrate 110 is larger than the refractive index of air, under the condition of total internal reflection, total internal reflection will happen to the beams transmitted toward the eyes of the operator U at the first interior surface SI1 of the first transparent substrate 110. As such, the ratio and the intensity of ultraviolet light IB transmitted to the eyes of the operator U is lowered, so as to increase the level of protection for the eyes of the operator U. However, the disclosure is not limited to the above. In another embodiment, an adhesive layer (e.g., an optical glue, not illustrated in FIG. 1) may be formed between the first transparent substrate 110 and the second transparent substrate 120, so as to attach the first transparent substrate 110 and the high reflection interference film 130 together using the adhesive layer.

Referring to FIG. 2, an eye protection device 100A is similar to the eye protection device 100 in FIG. 1, wherein the same elements are represented using the same labels and are not repeated here. The main differences between the eye protection device 100A and the eye protection device 100 in FIG. 1 are described as below. In the eye protection device 100A, the high reflection interference film 130 is disposed on the second exterior surface SO2 of the second transparent substrate 120, and there is no air gap AG as shown in FIG. 1 between the first transparent substrate 110 and the second transparent substrate 120. Specifically, the first transparent substrate 110 and the second transparent substrate 120 are in contact with each other, and the first transparent substrate 110 and the second transparent substrate 120 are, for example, fixed together in a detachable manner. For example, the first transparent substrate 110 and the second transparent substrate 120 are fixed together using a fixing mechanism, but the disclosure is not limited thereto. In another embodiment, the first transparent substrate 110 and the second transparent substrate 120 may also be fixed together using an adhesive layer (e.g., an optical glue, not illustrated in FIG. 2).

In yet another embodiment, the eye protection device 100A may further include two pieces of polarizers (e.g., high-intensity light adjustable polarizers, but are not limited thereto) to filter beams of specific polarization states and high-intensity beams. The two pieces of polarizers overlap the first transparent substrate 110 and the second transparent substrate 120. For example, the two pieces of polarizers may be attached onto at least one of the first exterior surface SO1 and the second exterior surface SO2 using an adhesive layer. When the polarizer and the high reflection interference film 130 are formed on the same surface of a transparent substrate, the high reflection interference film 130 may be formed on the surface first before attaching the polarizer, or attaching the polarizer first before forming the high reflection interference film 130. By rotating at least one of the two pieces of polarizers, an angle is formed between the absorption axes of the two pieces of polarizers to adjust (e.g., to lower) light intensity and glare. In an embodiment, at least one of the two pieces of polarizers may be fixed between the operator U and the laser processing machine using a clamping mechanism, and the at least one of the two pieces of polarizers does not have to be attached onto the first transparent substrate 110 or the second transparent substrate 120.

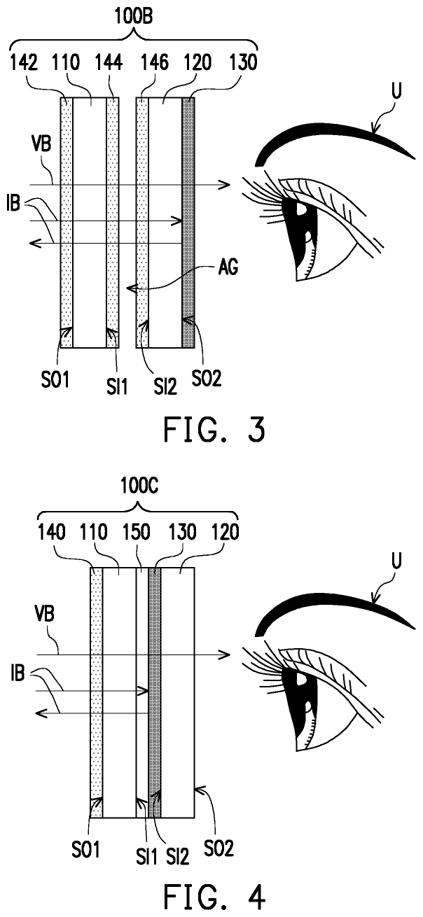

Referring to FIG. 3, an eye protection device 100B is similar to the eye protection device 100 in FIG. 1, wherein the same elements are represented using the same labels and are not repeated here. The main differences between the eye protection device 100B and the eye protection device 100 in FIG. 1 are described as below. In the eye protection device 100B, the high reflection interference film 130 is disposed on the second exterior surface SO2 of the second transparent substrate 120. In addition, the eye protection device 100B further includes a high transmittance anti-reflection film 142, a high transmittance anti-reflection film 144, and a high transmittance anti-reflection film 146. The high transmittance anti-reflection film 142 is disposed on the first exterior surface SO1 of the first transparent substrate 110. The high transmittance anti-reflection film 144 is disposed on the first interior surface SU of the first transparent substrate 110. The high transmittance anti-reflection film 146 is disposed on the second interior surface SI2 of the second transparent substrate 120. The high transmittance anti-reflection film 142, the high transmittance anti-reflection film 144, and the high transmittance anti-reflection film 146 are adapted to lower the interface reflection, which helps in improving the ghost image phenomenon, so as to increase clarity of viewing. For example, the high transmittance anti-reflection film 142, the high transmittance anti-reflection film 144, and the transmission anti-reflection film 146 may respectively be multilayer thin films. In an embodiment, the high transmittance anti-reflection film 142, the high transmittance anti-reflection film 144, and the high transmittance anti-reflection film 146 may also respectively be filters for filtering ultraviolet light. However, the filtering waveband of the filters may be selected according to the waveband of the harmful light source, and is not limited to ultraviolet light.

It should be noted that although the embodiment is exemplified using three high transmittance anti-reflection films, the quantity of the high transmittance anti-reflection films may change according to needs and is not limited to three. In another embodiment, at least one of the high transmittance anti-reflection film 142, the high transmittance anti-reflection film 144, and the high transmittance anti-reflection film 146 can be omitted.

Referring to FIG. 4, an eye protection device 100C is similar to the eye protection device 100 in FIG. 1, wherein the same elements are represented using the same labels and are not repeated here. The main differences between the eye protection device 100C and the eye protection device 100 in FIG. 1 are described as below. The eye protection device 100C further includes a high transmittance anti-reflection film 140. The high transmittance anti-reflection film 140 is disposed on the first exterior surface SO1 of the first transparent substrate 110, which is adapted to lower the interface reflection to help in improving the ghost image phenomenon, so as to increase clarity of viewing. The high transmittance anti-reflection film 140 may be a multilayer thin film. In an embodiment, the high transmittance anti-reflection film 140 may also be a filter for filtering ultraviolet light. However, the filtering waveband of the filter may be selected according to the waveband of the harmful light source and is not limited to ultraviolet light.

In addition, the eye protection device 100C may further include an adhesive layer 150. The adhesive layer 150 is positioned between the first transparent substrate 110 and the second transparent substrate 120, and the high reflection interference film 130 is, for example, positioned between the adhesive layer 150 and the second transparent substrate 120. The adhesive layer 150 may be formed only on the periphery of the first interior surface SI1. In another embodiment, the adhesive layer 150 may be omitted, and the first transparent substrate 110 and the second transparent substrate 120 may be fixed together using a fixing mechanism to increase convenience of element replacement (e.g., transparent substrate replacement).

In another embodiment, the eye protection device 100C may further include two pieces of polarizers (e.g., high-intensity light adjustable polarizers but are not limited to such) to filter beams of a specific polarization state and high-intensity beams. The two pieces of polarizers overlap the first transparent substrate 110 and the second transparent substrate 120. For example, the two pieces of polarizers may be attached onto at least one of the first interior surface SI1, the first exterior surface SO1, the second interior surface SI2, and the second exterior surface SO2 using an adhesive layer. When the polarizer and the high reflection interference film 130 (or the high transmittance anti-reflection film 140) are formed on the same surface of the transparent substrate, the high reflection interference film 130 (or the high transmittance anti-reflection film 140) may be formed on the surface first before attaching the polarizer, or attaching the polarizer first before forming the high reflection interference film 130 (or the high transmittance anti-reflection film 140). In addition, if the polarizer is formed on the interior surface of the transparent substrate, the polarizer may be attached first, then the first transparent substrate 110 and the second transparent substrate 120 are fixed together using the adhesive layer 150. By rotating at least one of the two pieces of polarizers, an angle is formed between the absorption axes of the two pieces of polarizers to adjust (e.g., to lower) light intensity and glare. In an embodiment, at least one of the two pieces of polarizers may be fixed between the operator U and the laser processing machine using a clamping mechanism, and the at least one of the two pieces of polarizers does not have to be attached onto the first transparent substrate 110 or the second transparent substrate 120.

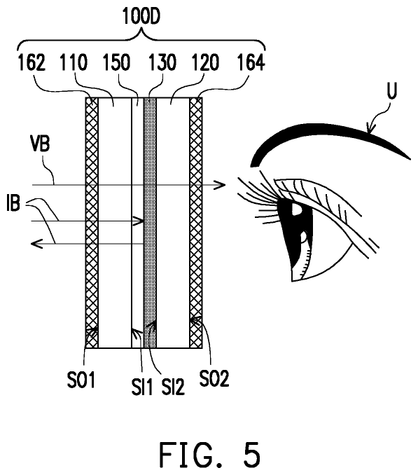

Referring to FIG. 5, an eye protection device 100D is similar to the eye protection device 100 in FIG. 1, wherein the same elements are represented using the same labels and are not repeated here. The main differences between the eye protection device 100D and the eye protection device 100 in FIG. 1 are described as below. The eye protection device 100D further includes the adhesive layer 150. The adhesive layer 150 is positioned between the first transparent substrate 110 and the second transparent substrate 120, and the high reflection interference film 130 is, for example, positioned between the adhesive layer 150 and the second transparent substrate 120. The adhesive layer 150 may be formed only on the periphery of the first interior surface SI1. In another embodiment, the adhesive layer 150 may be omitted, and the first transparent substrate 110 and the second transparent substrate 120 may be fixed together using a fixing mechanism to increase convenience of element replacement (e.g., transparent substrate replacement).

In addition, the eye protection device 100D may further include a protection layer 162 and a protection layer 164. The protection layer 162 is disposed on the first exterior surface SO1 while the protection layer 164 is disposed on the second exterior surface SO2. The protection layer 162 and the protection layer 164 are adapted to protect the first transparent substrate 110 and the second transparent substrate 120 and prevent the first transparent substrate 110 and the second transparent substrate 120 from being damaged (e.g., scratched). The material of the protection layer 162 and the protection layer 164 may be an inorganic insulation material. For example, the protection layer 162 and the protection layer 164 may be multilayer thin film protection layers composed of silicon carbide, silica, and silicon nitride, or the protection layer 162 and the protection layer 164 may be water resistant, abrasion resistant, and grease free multilayer structure protection layers formed by polymer combined with siloxy, but the disclosure is not limited thereto.

It should be noted that although this embodiment is exemplified using two protection layers, the quantity of the protection layers may change according to needs and is not limited to two. In other embodiments, at least one of the protection layer 162 and the protection layer 164 may be omitted.

In another embodiment, the eye protection device 100D can further include two pieces of polarizing plates (e.g., high-intensity light adjustable polarizing plates, but are not limited thereto) to filter beams of a specific polarization state and high-intensity beams. The two pieces of polarizers overlap the first transparent substrate 110 and the second transparent substrate 120. For example, the two pieces of polarizers may be attached onto at least one of the first interior surface SI1, the first exterior surface SO1, the second interior surface SI2, and the second exterior surface SO2 using an adhesive layer. When the polarizer and the high reflection interference film 130 (or the protection layers) are formed on the same surface of the transparent substrate, the high reflection interference film 130 (or the protection layers) may be formed on the surface first before attaching the polarizer, or attaching the polarizer first before forming the high reflection interference film 130 (or the protection layers). In addition, if the polarizer is formed on the interior surface (e.g., the first interior surface SI1) of the transparent substrate, the polarizer may be attached first, then the first transparent substrate 110 and the second transparent substrate 120 are fixed together using the adhesive layer 150. By rotating at least one of the two pieces of polarizers, an angle is formed between the absorption axes of the two pieces of polarizers to adjust (e.g., to lower) light intensity and glare. In an embodiment, at least one of the two pieces of polarizers may be fixed between the operator U and the laser processing machine using a clamping mechanism, and the at least one of the two pieces of polarizers does not have to be attached onto the first transparent substrate 110 or the second transparent substrate 120.

Based on the above, in the eye protection device of the disclosure, since the high reflection interference film is positioned between the first transparent substrate and the second transparent substrate, damages (e.g. scratches) caused to the high reflection interference film during cleaning of the eye protection device can be prevented, so as to maintain integrity of the high reflection interference film. In an embodiment, two pieces of polarizers can be disposed, and an angle is formed between the absorption axes of the two pieces of polarizers to adjust (e.g., to lower) light intensity and glare. In addition, by the arrangement of the two pieces of transparent substrates, a damaged transparent substrate can be replaced independently. Also, the high reflection interference film may be disposed on the transparent substrate which needs less replacements to decrease the number of replacements of the high reflection interference film, so as to lower the cost of element replacement.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An eye protection device, comprising:
a first transparent substrate;
a second transparent substrate opposite to the first transparent substrate; and
a high reflection interference film disposed on the first transparent substrate or the second transparent substrate.

2. The eye protection device according to claim 1, wherein the first transparent substrate and the second transparent substrate are respectively glass.

3. The eye protection device according to claim 1, wherein the high reflection interference film is positioned between the first transparent substrate and the second transparent substrate.

4. The eye protection device according to claim 1, wherein the high reflection interference film is a multilayer thin film allowing visible light to penetrate through and reflecting invisible light and light of laser processing wavelength.

5. The eye protection device according to claim 1, wherein air gap exists between the first transparent substrate and the second transparent substrate.

6. The eye protection device according to claim 1, further comprising:
an adhesive layer positioned between the first transparent substrate and the second transparent substrate.

7. The eye protection device according to claim 1, further comprising:
a high transmittance anti-reflection film disposed on at least one of the first transparent substrate and the second transparent substrate.

8. The eye protection device according to claim 7, wherein the high transmittance anti-reflection film is a filter for filtering invisible light and light of laser processing wavelength.

9. The eye protection device according to claim 1, wherein the first transparent substrate has a first interior surface facing the second transparent substrate and a first exterior surface opposite to the first interior surface, the second transparent substrate has a second interior surface facing the first transparent substrate and a second exterior surface opposite to the second interior surface, and the eye protection device further comprises:
protection layers disposed on the first exterior surface and the second exterior surface.

10. The eye protection device according to claim 1, wherein the eye protection device is protective goggles or an operation window of a laser processing machine.

11. The eye protection device according to claim 1, further comprising:
two pieces of polarizers overlapped the first transparent substrate and the second transparent substrate, and an angle is formed between absorption axes of the two pieces of polarizers.
